(19) United States
(12) Patent Application Publication
(10) Pub. No.: US 2006/0172397 A1
(43) Pub. Date: Aug. 3, 2006

(54) Preparation of chiral propargylic alcohol and ester intermediates of himbacine analogs

(75) Inventors: Tao Li, Scotch Plains, NJ (US); Maria M. Tamarez, Rahway, NJ (US); Aleksey Zaks, Hoboken, NJ (US)

Correspondence Address: SCHERING-PLOUGH CORPORATION;PATENT DEPARTMENT (K-6-1, 1990), 2000 GALLOPING HILL ROAD, KENILWORTH, NJ, 07033-0530, US

(73) Assignee: Schering Corporation

(21) Appl. No.: 11/330,936

(22) Filed: Jan. 12, 2006

Related U.S. Application Data
(60) Provisional application No. 60/643,927, filed on Jan. 14, 2005.

(51) Int. Cl. C12P 7/62; C07C 69/74

(52) U.S. Cl. 435/135; 560/128

(57) ABSTRACT

This application discloses a novel process for the conversion of a series of racemic propargylic alcohols to corresponding (R)-enantiomers. The application also discloses the enantio-selective esterification of a propargylic alcohol from its racemate to prepare an (R)-ester. Enantioselectivity is enhanced by the use of experimentally determined enzymes. The propargylic alcohols and chiral esters may be useful in preparing compounds such as, for example, thrombin receptor antagonists. Among the synthetic pathways disclosed is the following:

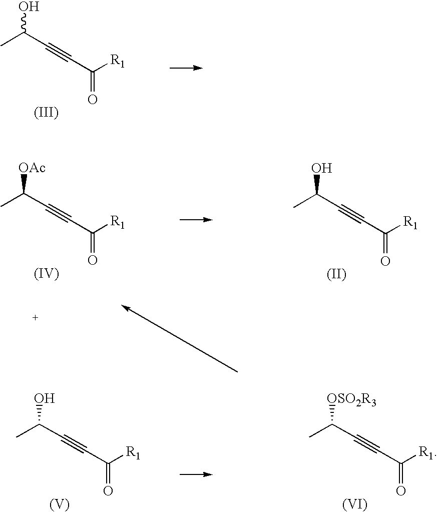

This application claims the benefit of U.S. provisional application Ser. No. 60/643,927 filed Jan. 14, 2005.

### FIELD OF THE INVENTION

This application discloses a novel process for the conversion of a series of racemic propargylic alcohols to corresponding (R)-enantiomers. The application also discloses the enantio-selective esterification of a propargylic alcohol from its racemate to prepare an (R)-ester. The propargylic alcohols and chiral esters may be useful in preparing compounds such as, for example, thrombin receptor antagonists. The invention disclosed herein is related to those disclosed in the co-pending patent applications corresponding to U.S. provisional application Ser. Nos. 60/643,932, 60/644,464, 60/644,428, all four applications having been filed on the same date.

### BACKGROUND OF THE INVENTION

Thrombin is known to have a variety of activities in different cell types and thrombin receptors are known to be present in such cell types as human platelets, vascular smooth muscle cells, endothelial cells, and fibroblasts. Thrombin receptor antagonists may be useful in the treatment of thrombotic, inflammatory, atherosclerotic and fibroproliferative disorders, as well as other disorders in which thrombin and its receptor play a pathological role. See, for example, U.S. Pat. No. 6,063,847, the disclosure of which is incorporated by reference.

In view of the importance of thrombin receptor antagonists, new methods for preparing such compounds that are both scalable and efficient are always of interest. Processes for the synthesis of similar himbacine analog thrombin receptor antagonists are disclosed in U.S. Pat. No. 6,063,847, and U.S. publication no. 2004/0216437A1, and the synthesis of the bisulfate salt of a particular himbacine analog is disclosed in U.S. publication no. 2004/0176418A1, the disclosures of which are incorporated by reference herein.

### SUMMARY OF THE INVENTION

In an embodiment, the present application teaches a novel, simple enantioselective process of making a compound of Formula (I) from a compound of formula (II):

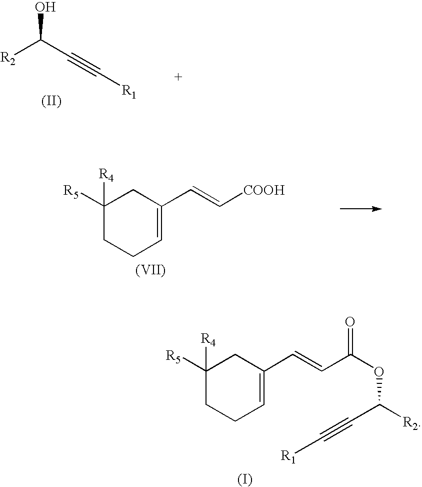

The process of making (I) from (II) comprises:
(a) reacting a compound of formula (III): 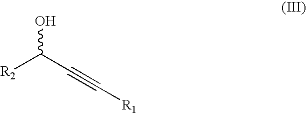
with a carboxylic ester, preferably acetate, in the presence of a resolving enzyme to yield compounds of formulae (IV) and (V): 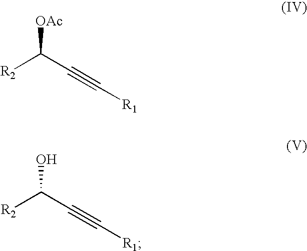
(b) sulfonating the compound of formula (V) to yield a sulfonate compound of formula (VI): 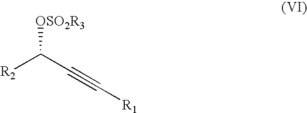
said sulfonate compound of formula (VI) being either removed by washing with water or converted to acetate compound of formula (IV) by displacement of sulfonate group to acetate group;
(c) converting the compound of formula (IV) to the compound of formula (II); and
(d) esterifying a compound of formula (VII): 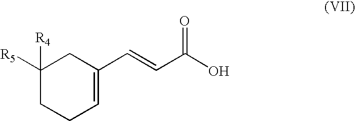
with the compound of formula (II) to yield the compound of formula (I),

where R₁ and R₂ are each independently selected from the group consisting of hydrogen, halogen, alkyl, haloalkyl, alkoxy, mono- and di-alkoxyalkyl, alkenyl, alkynyl, mono- and di-alkylamino, mono- and di-arylamino, (aryl)alkylamino, (alkyl)arylamino, amido, mono- and di-alkylamido, and mono- and di-arylamido groups;

R₃ is selected from the group consisting of alkyl, aryl, arylalkyl, and heteroaryl groups;

R₄ and R₅ are each independently selected from the group consisting of H, hydroxyl, amino, nitro, amido, halogen, alkyl, alkenyl, alkoxy, mon- and di-alkoxyalkyl-, alkoxyalkyl, halo(C₁-C₆ alkyl)-, dihaloalkyl-, trihaloalkyl-, cycloalkyl, cycloalkyl-alkyl-, aryl, alkyl-aryl, aryl-alkyl-, thioalkyl, alkyl-thioalkyl, alkenyl, hydroxyl-alkyl-, aminoalkyl-, —C(O)OR₇, —C(O)NR₈R₉, -alkyl-C(O)NR₈R₉, —NR₁₀R₁₁, and N₁₀R₁₁-alkyl, or R₄ and R₅, together with the carbon to which they are attached, form a heteroaryl or heterocyclic group of 5 to 10 atoms comprised of hydrogen atoms, 1 to 9 carbon atoms, and 1 to 4 heteroatoms independently selected from the group consisting of N, O, and S, wherein a ring nitrogen can form an N-oxide or a quaternary group with a (C₁-C₄)alkyl group;

R₇, R₈, and R₉ are each independently selected from the group consisting of H, (C₁-C₆)alkyl, phenyl, and benzyl; and

R₁₀ and R₁₁ are each independently selected from the group consisting of H and (C₁-C₆)alkyl.

It is to be noted that the conversion of the sulfonate compound of formula (VI) to the acetate compound of formula (IV) by displacement of sulfonate group to acetate group involves an inversion.

The compound of formula (I) can also be prepared from the compound of formula (VII) by a process comprising:
(a) activating a compound of formula (VII) to yield a compound of formula (VIII): 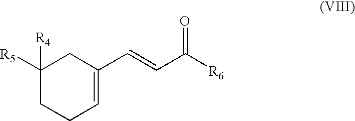
(b) reacting the compound of formula (VIII), in the presence of an enzyme, with a compound of formula (III): 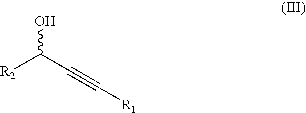
where R₁, R₂, R₄ and R₅ are as defined above, and R₆ is selected from the group consisting of alkoxy and alkenyloxy, each of which may be unsubstituted or substituted with at least one of halogen atoms and nitro, amino, and (C₁-C₆)alkoxy groups, ONH₂, ONH(CₙH₂ₙ₊₁), ON(CₙH₂ₙ₊₁)(CₙH₂ₙ), ON(CₙH₂ₙ), and ON(CₙH₂ₙ₊₁)₂, wherein n ranges from 1 to 6;

In another embodiment, the compound of formula (II) can be prepared by a process comprising: (a) reacting a compound of formula (III) with an acetate in the presence of a resolving enzyme to yield compounds of formulae (IV) and (V):

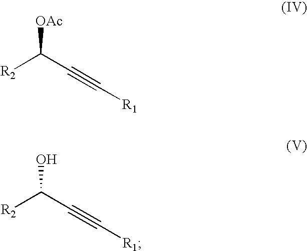

(b) sulfonating the compound of formula (V) to yield a compound of formula (VI):

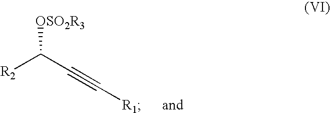

(c) converting the compound of formula (IV) to the compound of formula (II), wherein R₁, R₂ and R₃ are as defined above.

It is to be understood that both the foregoing general description and the following description of various embodiments are exemplary and explanatory only and are not restrictive.

### DESCRIPTION OF THE INVENTION

A thrombin receptor antagonist of particular interest is a compound of formula (IX):

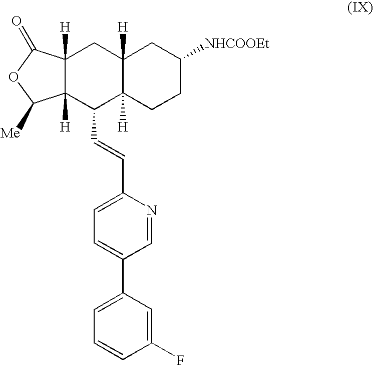

This compound is an orally bioavailable thrombin receptor antagonist derived from himbacine. The tricyclic motif of compound (IX) may be prepared from (R)-propargylic alcohol (II) and ester (I) from the following scheme:

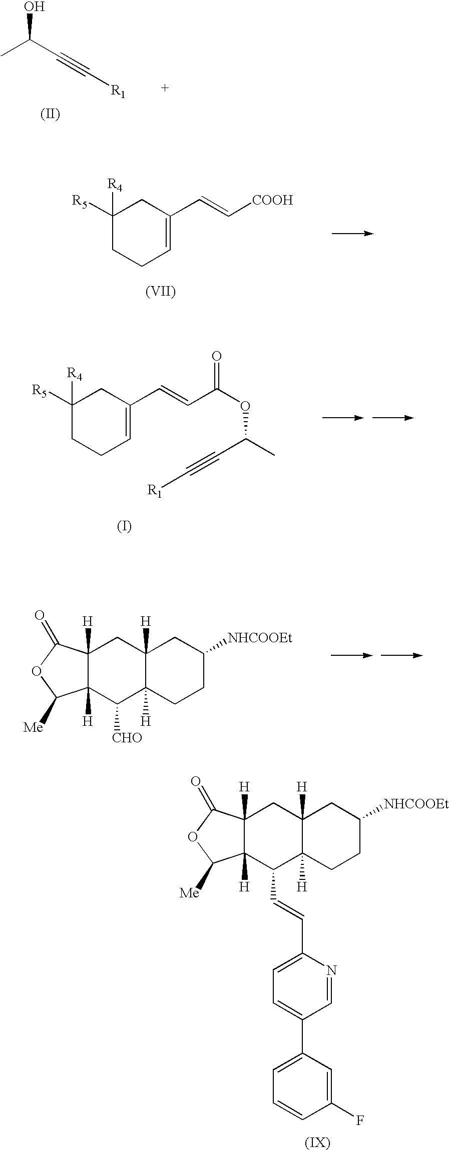

where R₁ is selected from the group consisting of hydrogen, halogen, alkyl, haloalkyl, alkoxy, mono- and di-alkoxyalkyl, alkenyl, alkynyl, mono- and di-alkylamino, mono- and di-arylamino, (aryl)alkylamino, (alkyl)arylamino, amido, mono- and di-alkylamido, and mono- and di-arylamido groups;

R₄ and R₅ are each independently selected from the group consisting of H, hydroxyl, amino, nitro, amido, halogen, alkyl, alkenyl, alkoxy, mon- and di-alkoxyalkyl-, alkoxyalkyl, halo(C₁-C₆ alkyl)-, dihaloalkyl-, trihaloalkyl-, cycloalkyl, cycloalkyl-alkyl-, aryl, alkyl-aryl, aryl-alkyl-, thioalkyl, alkyl-thioalkyl, alkenyl, hydroxyl-alkyl-, aminoalkyl-, —C(O)OR₇, —C(O)NR₈R₉, -alkyl-C(O)NR₈R₉, —NR₁₀R₁₁, and N₁₀R₁₁-alkyl, or R₄ and R₅, together with the carbon to which they are attached, form a heteroaryl or heterocyclic group of 5 to 10 atoms comprised of hydrogen atoms, 1 to 9 carbon atoms, and 1 to 4 heteroatoms independently selected from the group consisting of N, O, and S, wherein a ring nitrogen can form an N-oxide or a quaternary group with a (C₁-C₄)alkyl group;

R₇, R₅, and R₉ are each independently selected from the group consisting of H, (C₁-C₆)alkyl, phenyl, and benzyl; and

R₁₀ and R₁₁ are each independently selected from the group consisting of H and (C₁-C₆)alkyl.

Racemic propargylic alcohols can be resolved by enzymes, for example lipases, or microorganisms, providing moderate to high enantioselectivity. After lipase resolution, the products may be recovered by separating the ester of one enantiomer from the alcohol of the opposite enantiomer. However, the separation of an alcohol from its ester can be difficult to scale up, and the yields of the product will generally be less than 50% because the opposite enantiomers are discarded.

The following definitions and terms are used herein or are otherwise known to a skilled artisan. Except where stated otherwise, the definitions apply throughout the specification and claims. Chemical names, common names and chemical structures may be used interchangeably to describe the same structure. These definitions apply regardless of whether a term is used by itself or in combination with other terms, unless otherwise indicated. Hence, the definition of “alkyl” applies to “alkyl” as well as the “alkyl” portions of “hydroxyalkyl,” “haloalkyl,” “alkoxy,” etc.

Unless otherwise known, stated or shown to be to the contrary, the point of attachment for a multiple term substituent (two or more terms that are combined to identify a single moiety) to a subject structure is through the last named term of the multiple term substituent. For example, a cycloalkylalkyl substituent attaches to a targeted structure through the latter “alkyl” portion of the substituent (e.g., structure-alkyl-cycloalkyl).

The identity of each variable appearing more than once in a formula may be independently selected from the definition for that variable, unless otherwise indicated.

Unless stated, shown or otherwise known to be the contrary, all atoms illustrated in chemical formulas for covalent compounds possess normal valencies. Thus, hydrogen atoms, double bonds, triple bonds and ring structures need not be expressly depicted in a general chemical formula.

Double bonds, where appropriate, may be represented by the presence of parentheses around an atom in a chemical formula. For example, a carbonyl functionality, —CO—, may also be represented in a chemical formula by —C(O)— or —C(=0)—. Similarly, a double bond between a sulfur atom and an oxygen atom may be represented in a chemical formula by —SO—, —S(O)— or —S(=0)—. One skilled in the art will be able to determine the presence or absence of double (and triple bonds) in a covalently-bonded molecule. For instance, it is readily recognized that a carboxyl functionality may be represented by —COOH, —C(O)OH, —C(═O)OH or —CO₂H.

The term “substituted,” as used herein, means the replacement of one or more atoms or radicals, usually hydrogen atoms, in a given structure with an atom or radical selected from a specified group. In the situations where more than one atom or radical may be replaced with a substituent selected from the same specified group, the substituents may be, unless otherwise specified, either the same or different at every position. Radicals of specified groups, such as alkyl, cycloalkyl, heterocycloalkyl, aryl and heteroaryl groups, independently of or together with one another, may be substituents on any of the specified groups, unless otherwise indicated.

The term “substituted or unsubstituted” means, alternatively, not substituted or substituted with the specified groups, radicals or moieties. It should be noted that any atom with unsatisfied valences in the text, schemes, examples and tables herein is assumed to have the hydrogen atom(s) to satisfy the valences.

The term “chemically-feasible” is usually applied to a ring structure present in a compound and means that the ring structure (e.g., the 4- to 7-membered ring, optionally substituted by . . . ) would be expected to be stable by a skilled artisan.

The term “heteroatom,” as used herein, means a nitrogen, sulfur or oxygen atom. Multiple heteroatoms in the same group may be the same or different.

As used herein, the term “alkyl” means an aliphatic hydrocarbon group that can be straight or branched and comprises 1 to about 24 carbon atoms in the chain. Preferred alkyl groups comprise 1 to about 15 carbon atoms in the chain. More preferred alkyl groups comprise 1 to about 6 carbon atoms in the chain. “Branched” means that one or more lower alkyl groups such as methyl, ethyl or propyl, are attached to a linear alkyl chain. The alkyl can be substituted by one or more substituents independently selected from the group consisting of halo, aryl, cycloalkyl, cyano, hydroxy, alkoxy, alkylthio, amino, —NH(alkyl), —NH(cycloalkyl), —N(alkyl)₂ (which alkyls can be the same or different), carboxy and —C(O)O-alkyl. Non-limiting examples of suitable alkyl groups include methyl, ethyl, n-propyl, isopropyl, n-butyl, t-butyl, n-pentyl, heptyl, nonyl, decyl, fluoromethyl, trifluoromethyl and cyclopropylmethyl.

“Alkenyl” means an aliphatic hydrocarbon group (straight or branched carbon chain) comprising one or more double bonds in the chain and which can be conjugated or unconjugated. Useful alkenyl groups can comprise 2 to about 15 carbon atoms in the chain, preferably 2 to about 12 carbon atoms in the chain, and more preferably 2 to about 6 carbon atoms in the chain. The alkenyl group can be substituted by one or more substituents independently selected from the group consisting of halo, alkyl, aryl, cycloalkyl, cyano and alkoxy. Non-limiting examples of suitable alkenyl groups include ethenyl, propenyl, n-butenyl, 3-methylbut-enyl and n-pentenyl.

Where an alkyl or alkenyl chain joins two other variables and is therefore bivalent, the terms alkylene and alkenylene, respectively, are used.

“Alkoxy” means an alkyl-O— group in which the alkyl group is as previously described. Useful alkoxy groups can comprise 1 to about 12 carbon atoms, preferably 1 to about 6 carbon atoms. Non-limiting examples of suitable alkoxy groups include methoxy, ethoxy and isopropoxy. The alkyl group of the alkoxy is linked to an adjacent moiety through the ether oxygen.

The term “cycloalkyl” as used herein, means an unsubstituted or substituted, saturated, stable, non-aromatic, chemically-feasible carbocyclic ring having preferably from three to fifteen carbon atoms, more preferably, from three to eight carbon atoms. The cycloalkyl carbon ring radical is saturated and may be fused, for example, benzofused, with one to two cycloalkyl, aromatic, heterocyclic or heteroaromatic rings. The cycloalkyl may be attached at any endocyclic carbon atom that results in a stable structure. Preferred carbocyclic rings have from five to six carbons. Examples of cycloalkyl radicals include cyclopropyl, cyclobutyl, cyclopentyl, cyclohexyl, cycloheptyl, or the like.

The term “alkenyl,” as used herein, means an unsubstituted or substituted, unsaturated, straight or branched, hydrocarbon chain having at least one double bond present and, preferably, from two to fifteen carbon atoms, more preferably, from two to twelve carbon atoms.

“Alkynyl” means an aliphatic hydrocarbon group comprising at least one carbon-carbon triple bond and which may be straight or branched and comprising about 2 to about 15 carbon atoms in the chain. Preferred alkynyl groups have about 2 to about 10 carbon atoms in the chain; and more preferably about 2 to about 6 carbon atoms in the chain. Branched means that one or more lower alkyl groups such as methyl, ethyl or propyl, are attached to a linear alkynyl chain. Non-limiting examples of suitable alkynyl groups include ethynyl, propynyl, 2-butynyl, 3-methylbutynyl, n-pentynyl, and decynyl. The alkynyl group may be substituted by one or more substituents which may be the same or different, each substituent being independently selected from the group consisting of alkyl, aryl and cycloalkyl.

The term “aryl,” as used herein, means a substituted or unsubstituted, aromatic, mono- or bicyclic, chemically-feasible carbocyclic ring system having from one to two aromatic rings. The aryl moiety will generally have from 6 to 14 carbon atoms with all available substitutable carbon atoms of the aryl moiety being intended as possible points of attachment. Representative examples include phenyl, tolyl, xylyl, cumenyl, naphthyl, tetrahydronaphthyl, indanyl, indenyl, or the like. If desired, the carbocyclic moiety can be substituted with from one to five, preferably, one to three, moieties, such as mono- through pentahalo, alkyl, trifluoromethyl, phenyl, hydroxy, alkoxy, phenoxy, amino, monoalkylamino, dialkylamino, or the like.

“Heteroaryl” means a monocyclic or multicyclic aromatic ring system of about 5 to about 14 ring atoms, preferably about 5 to about 10 ring atoms, in which one or more of the atoms in the ring system is/are atoms other than carbon, for example nitrogen, oxygen or sulfur. Mono- and polycyclic (e.g., bicyclic) heteroaryl groups can be unsubstituted or substituted with a plurality of substituents, preferably, one to five substituents, more preferably, one, two or three substituents (e.g., mono-through pentahalo, alkyl, trifluoromethyl, phenyl, hydroxy, alkoxy, phenoxy, amino, monoalkylamino, dialkylamino, or the like). Typically, a heteroaryl group represents a chemically-feasible cyclic group of five or six atoms, or a chemically-feasible bicyclic group of nine or ten atoms, at least one of which is carbon, and having at least one oxygen, sulfur or nitrogen atom interrupting a carbocyclic ring having a sufficient number of pi (π) electrons to provide aromatic character. Representative heteroaryl (heteroaromatic) groups are pyridinyl, pyrimidinyl, pyrazinyl, pyridazinyl, furanyl, benzofuranyl, thienyl, benzothienyl, thiazolyl, thiadiazolyl, imidazolyl, pyrazolyl, triazolyl, isothiazolyl, benzothiazolyl, benzoxazolyl, oxazolyl, pyrrolyl, isoxazolyl, 1,3,5-triazinyl and indolyl groups.

The term “heterocyclic ring” or “heterocycle,” as used herein, means an unsubstituted or substituted, saturated, unsaturated or aromatic, chemically-feasible ring, comprised of carbon atoms and one or more heteroatoms in the ring. Heterocyclic rings may be monocyclic or polycyclic. Monocyclic rings preferably contain from three to eight atoms in the ring structure, more preferably, five to seven atoms. Polycyclic ring systems consisting of two rings preferably contain from six to sixteen atoms, most preferably, ten to twelve atoms. Polycyclic ring systems consisting of three rings contain preferably from thirteen to seventeen atoms, more preferably, fourteen or fifteen atoms. Each heterocyclic ring has at least one heteroatom. Unless otherwise stated, the heteroatoms may each be independently selected from the group consisting of nitrogen, sulfur and oxygen atoms.

The term “hydroxyl alkyl,” as used herein, means a substituted hydrocarbon chain preferably an alkyl group, having at least one hydroxy substituent (-alkyl-OH). Additional substituents to the alkyl group may also be present. Representative hydroxyalkyl groups include hydroxymethyl, hydroxyethyl and hydroxypropyl groups.

The terms “Hal,” “halo,” “halogen” and “halide,” as used herein, mean a chloro, bromo, fluoro or iodo atom radical. Chlorides, bromides and fluorides are preferred halides.

The term “phase transfer catalyst,” as used herein, means a material that catalyzes a reaction between a moiety that is soluble in a first phase, e.g., an organic phase, and another moiety that is soluble in a second phase, e.g., an aqueous phase.

The following abbreviations are used in this application: ee is enantiomeric excess; de is diastereomeric excess; EtOH is ethanol; Me is methyl; Et is ethyl; Bu is butyl; n-Bu is normal-butyl, t-Bu is tert-butyl, OAc is acetate; KOt-Bu is potassium tert-butoxide; MeCN is acetonitrile; TBME is tert-butyl methyl ether; NBS is N-bromo succinimide; NMP is 1-methyl-2-pyrrolidinone; DMA is N,N-dimethylacetamide; n-Bu₄NBr is tetrabutylammonium bromide; n-Bu₄NOH is tetrabutylammonium hydroxide, n-Bu₄NH₂SO₄ is tetrabutylammonium hydrogen sulfate, and equiv. is equivalents.

### General Syntheses

A practical route was discovered to convert racemic propargylic alcohols (III) to (II) in 100% theoretical yield. In this strategy, racemic alcohols (III) were resolved by a lipase in the presence of an acetate to give (V) and (IV). Subsequently, (V) was activated by forming sulfonate (VI) followed by chiral inversion. The chiral inversion of (VI) was achieved by acetate displacement to give (IV). The acetate (IV) was then converted to alcohol (II) by methanolysis under basic conditions, or by enzyme hydrolysis. The overall yields were between 70% to 80%, and the ee for (II) were between 96% and 98%.

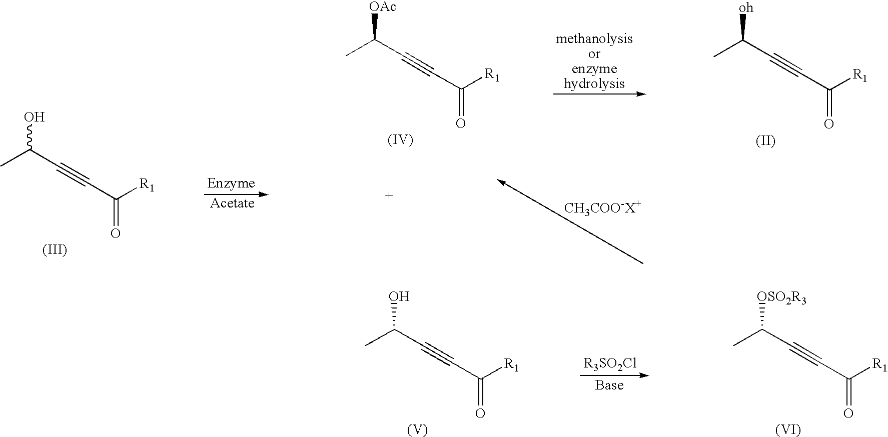

Step 1—Enzyme Resolution: The enzyme resolution may be performed with a lipase in the presence of a carboxylic ester, preferably an acetate and a solvent. Suitable acetates include alkyl and alkenyl acetates, such as, for example, ethyl acetate, isopropenyl acetate, vinyl acetate and the like. Preferably, vinyl acetate is used. Suitable solvents include organic solvents. Preferred solvents are TBME and MeCN. A number of enzymes are suitable for resolving (III) to (IV) and (V). A lipase is preferred. Table 1 identifies enzymes that can resolve (III) when R₁ is CH(OEt)₂.

TABLE 1

| Enzyme | Source | Solvent |
|---|---|---|
| Lipase PS | Amano | TBME |
| Lipase AK | Amano | TBME |
| Lipase PS-C | Amano | MeCN |
| Lipase PS-D | Amano | MeCN |
| Chirazyme L-7 | Biocatalytica/Roche | TBME |
| Chirazyme L-9 | Biocatalytica/Roche | TBME |
| Lipase BC | Biocatalytica/Roche | TBME |
| ICR107 | Biocatalytica/Roche | TBME |
| ICR108 | Biocatalytica/Roche | TBME |
| ICR109 | Biocatalytica/Roche | TBME |
| Lipase 20 | Europa | MeCN |
| Lipase 11 | Europa | MeCN |
| Lipase 4 | Europa | MeCN |
| Lipase 3 | Europa | MeCN |
| Lipase 21 | Europa | MeCN |
| Lipase CE | Sci. Protein Labs | TBME |
| High Lipase PEC | Sci. Protein Labs | TBME |
| Lipase Type II | Sigma/Fluka | TBME |
| Steapsin | | |
| Lipase LIP-300 | Toyobo | MeCN |

Table 2 illustrates the resolving enzymes that can resolve (III) when R₁ is C(O)N(PH)₂:

TABLE 2

| Resolving Enzyme | Vendor | Solvent |
|---|---|---|
| Lipase PS | Amano | MeCN |
| Lipase PS-C | Amano | MeCN |
| Lipase PS-D | Amano | MeCN |
| Chirazyme L-7 | Roche | TBME |
| Chirazyme L-9 | Biocatalytica/Roche | TBME |
| Lipase BC | Biocatalytica/Roche | TBME |
| ICR108 | Biocatalytica/Roche | MeCN |
| Lipase 20 | Europa | MeCN |
| Lipase 4 | Europa | MeCN |
| Lipase 3 | Europa | MeCN |
| Lipase 21 | Europa | MeCN |
| Porcine pancreat | K-P/Biocatalysts | TBME |
| Lipase | | |
| High Lipase PEC | Sci. Protein Labs | TBME |
| Lipase Type II | Sigma/Fluka | TBME |
| steapsin | | |
| Fungal esterase ISC- | Interspex | TBME |
| 03_FE1 | | |
| Penicillium Acylase | Julich | TBME |

Step 2—Sulfonation: The sulfonation of (V) to (VI) is preferably conducted under typical sulfonation conditions known to those skilled in the art. According to various embodiments, a suitable sulfonating agent is of the formula R₃SO₂X, wherein R₃ is selected from the group consisting of alkyl, aryl, arylalkyl, and heteroaryl groups, and X is halogen. Another suitable sulfonating agent is SO₃.Pyr. Suitable bases include pyridine, triethylamine, 1,4-diazabicyclo[2,2,2]octane, Hoenigs base and the like. According to various embodiments, the sulfonation is conducted in the same pot in which the enzymatic resolution was conducted, preferably after at least a portion of the enzyme is removed.

Step 3—Sulfonate Displacement: Sulfonate (VI) may be converted to acetate (IV) by displacement. The enantioselective conversion may be achieved in a multiphasic system in the presence of a phase transfer catalyst and a carboxylate salt, such as, for example, potassium acetate, or in a monophasic system in the presence of a nucleophile, such as tetratebutylammonium acetate. In each case the ee may be fully retained, with a yield ranging from 65% to 90%.

Step 4—Acetate Deprotection: The acetate (IV) may be deprotected to (II) by alcoholysis, for example methanolysis, under basic conditions. The base could be, for example, sodium or potassium carbonate. The reaction may be facilitated in the presence of a phase transfer catalyst. In this step, the ee of (II) may be fully retained, with a yield typically of 90%. Alternatively, the deprotection of the acetate can also be carried out by enzyme hydrolysis. Typically, the reaction gave (II) in >90% yield and >98% ee.

Another embodiment of the present application relates to enantioselective esterification of an alcohol from its racemate to prepare ester (I):

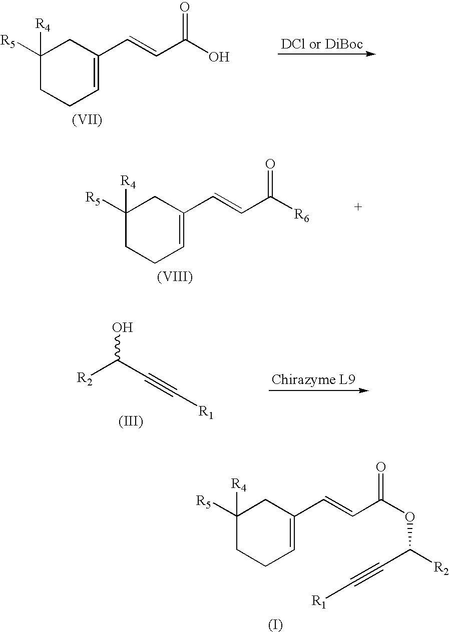

Ester (I) is an intermediate in the synthesis of compound (IX), supra A practical method was discovered for preparing enantio-pure (I) starting from the acid (VII) and racemic alcohol (III) by lipase catalyzed coupling. In this method, the acid (VII) is activated to give the corresponding ester (VIII) in nearly quantitative yield. The ester is then coupled with the (R) enantiomer of the racemate (III) in the presence of an enzyme to give enantiomerically enriched (I).

The lipases were found to carry out the (R) enantioselective coupling. These enzymes included Chirazyme L-9 (Biocatalytica/Roche), Mucor miehei lipase (Enzeco), and Cholesterol esterase (Amano). Under optimal conditions, Chirazyme L-9 was able to catalyze the coupling of (VIII) with (R)-(III) efficiently to give (I) with >98% ee. In these instances, R₆ is O—N═C(Me)₂. A summary is provided in Table 3.

TABLE 3

| | | | | | | | De or |
|---|---|---|---|---|---|---|---|
| R₁ | R₂ | R₄ | R₅ | R₆ | Hours | Conv. % | ee % for (I) |
| CH(OEt)₂ | Me | OCH₂CH₂O | OCH₂CH₂O | O—N═C(Me)₂ | 44 | 98 | >99 |
| CH(OEt)₂ | Me | NHCOOEt | H | O—N═C(Me)₂ | 72 | 65 | >99 |
| CONPh₂ | Me | NHCOOEt | H | O—N═C(Me)₂ | 24 | 100 | 98.5 |
| CH(OEt)₂ | Me | NO₂ | H | O—N═C(Me)₂ | 68 | 95 | 99 |
| CONPh₂ | Me | NO₂ | H | O—N═C(Me)₂ | 24 | 100 | 98.9 |

### EXAMPLES

### Example 1

### Screen for Enzymes to Resolve (III)

The following scheme was used to screen for enzymes suitable for resolving (III) to (IV) and (V):

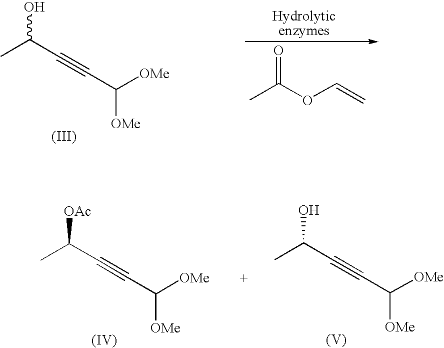

The reaction mixture contained 10 mg (III), 60 mg of vinyl acetate, and 10 mg of an enzyme in 1 ml solvent. The solvent was either MeCN or TBME. The reactions were carried out by agitation at 25° C. After 24 h, the reaction mixture was subjected to analysis of (III) and the corresponding acetate (IV) by the following method:

GC with FID Detection

| | |
|---|---|
| Column: | β-dex 110 (Supelco), 30 m × 0.25 mm × 0.25μ |
| Carrier gas: | Helium 1 ml/min |
| Inlet: | 180° C. |
| Split ratio: | 1:100 |
| Oven temperature: | isothermal at 100° C. |
| Retention time: | |
| (R)-III | 30.8 min |
| (S)-III | 31.9 min |
| (R)-IV | 35.3 min |
| (S)-IV | 34.4 min |

In total, 212 commercially available enzymes were tested. These enzymes included 85 lipases, 95 proteases or peptidases, 10 amidases or acylases, and 22 esterases. 52 enzymes, including 46 lipases, 2 acylases, and 4 esterases were found to be R selective. Among them, 15 lipases showed very high R selectivity (with E>200). There were 3 proteases that exhibited moderate S selectivity. The results of the lipases with high R selectivity and proteases with S selectivity are summarized in Table 4.

TABLE 4

| | | | | ee for | ee for | |
|---|---|---|---|---|---|---|
| | | | | 5a | 4a | |
| Enzyme | Vendor | Solvent | Conversion % | (Config.) | (Config.) | E |
| Lipase PS | Amano | TBME | 50 | 99.3 | 99.5 | >200 |
| | | | | (S) | (R) | |
| Lipase AK | Amano | TBME | 50 | 99.4 | 99.5 | >200 |
| | | | | (S) | (R) | |
| Lipase PS-C | Amano | MeCN | 50 | 99.3 | 99.4 | >200 |
| | | | | (S) | (R) | |
| Lipase PS-D | Amano | MeCN | 50 | 99.2 | 99.4 | >200 |
| | | | | (S) | (R) | |
| Lipase BC | BioCatalytics | TBME | 49 | 95.8 | 99.6 | >200 |
| | | | | (S) | (R) | |
| Lipase ICR- | BioCatalytics | TBME | 50 | 99.4 | 99.5 | >200 |
| 107 | | | | (S) | (R) | |
| Lipase ICR- | BioCatalytics | TBME | 50 | 99.4 | 99.5 | >200 |
| 108 | | | | (S) | (R) | |
| Europa | Europa | MeCN | 50 | 99.3 | 99.4 | >200 |
| Lipase 20 | Bioproducts | | | (S) | (R) | |
| Europa | Europa | MeCN | 50 | 99.2 | 99.4 | >200 |
| Lipase 4 | Bioproducts | | | (S) | (R) | |
| Europa | Europa | MeCN | 48.7 | 94.4 | 99.5 | >200 |
| lipase 3 | Bioproducts | | | (S) | (R) | |
| Europa | Europa | TBME | 50 | 99.4 | 99.6 | >200 |
| Lipase 21 | Bioproducts | | | (S) | (R) | |
| Lipase LIP- | Toyobo | MeCN | 50 | 99.3 | 99.5 | >200 |
| 300 | | | | (S) | (R) | |
| Chlesterol | Amano | MeCN | 49.1 | 96.1 | 99.5 | >200 |
| esterase | | | | (S) | (R) | |
| Lipase AH | Amano | TBME | 46.3 | 85.8 | 99.6 | >200 |
| | | | | (S) | (R) | |
| ICS-04-BP1 | Interspex | MeCN | 35.6 | 36.1 | 65.2 | 7 |
| | | | | (R) | (S) | |
| ICS-04-BP2 | Interspex | MeCN | 14.3 | 13.1 | 78.2 | 9 |
| | | | | (R) | (S) | |
| ICS-01-BP2 | Interspex | MeCN | 18.7 | 18.2 | 79.1 | 10 |
| | | | | (R) | (S) | |

### Example 2

### Screen for Enzymes to Resolve (III)

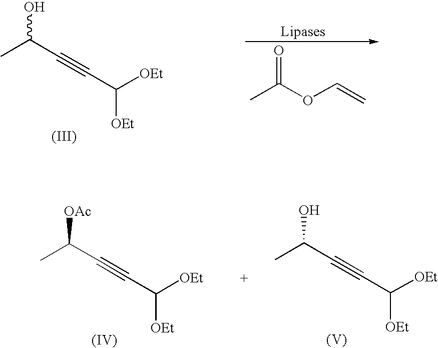

In order to find the most efficient enzyme for the resolution, the screen was conducted with a set of 8 lipases at high concentration (1 M) of (III). The lipases in the set were picked from those which were selective for (III) in Example 1.

The reaction mixture contained 172 mg of (III), 185 mg of vinyl acetate, 10 mg of a lipase, and 1 ml of a solvent. The solvent was either TBME or MeCN. After 24 h, the enzyme was filtered and the reaction mixture was analyzed with the following method for (III) and the corresponding acetates (IV):

GC with FID detection

| | |
|---|---|
| Column: | β-dex 110 (Supelco), 30 m × 0.25 mm × 0.25μ |
| Carrier gas: | Helium 1.5 ml/min |
| Inlet: | 180° C. |
| Split ratio: | 1:100 |
| Oven temperature: | isothermal at 95° C. |
| Retention time: | |
| (R)-(III) | 50.8 min |
| (S)-(III) | 51.8 min |
| (R)-(IV) | 66.5 min |
| (S)-(IV) | 64.4 min |

All lipases remained highly R selective, but they had different activities for (III) (see Table 5). Europa Lipase 20 turned out to be the most active lipase.

Table 5—Enzymes Identified to Resolve (III) by Acylation with Vinyl Acetate

TABLE 5

| | | | | | ee for | |
|---|---|---|---|---|---|---|
| | | | Conver- | ee for 5b | 4b | |
| Enzyme | Vendor | Solvent | sion % | (config.) | (config.) | E |
| Lipase | Amano | TBME | 20.4 | 25.5 (S) | >99 R | >200 |
| PS | | | | | | |
| Lipase | Amano | TBME | 32.8 | 48.6 (S) | >99 R | >200 |
| AK | | | | | | |
| Lipase | Europa | MeCN | 50 | 98.4 (S) | >99 R | >200 |
| 20 | | | | | | |
| Lipase 4 | Europa | MeCN | 22.4 | 28.7 (S) | >99 R | >200 |
| Lipase | Europa | TBME | 43.3 | 76.1 (S) | >99 R | >200 |
| 21 | | | | | | |
| LIP300 | Toyobo | MeCN | 16.8 | 20.1 (S) | >99 R | >200 |
| Lipase | Amano | TBME | 13 | 14.9 (S) | >99 R | >200 |
| AH | | | | | | |

### Example 3

### Multi-Gram Resolution of (II) with Lipase 20

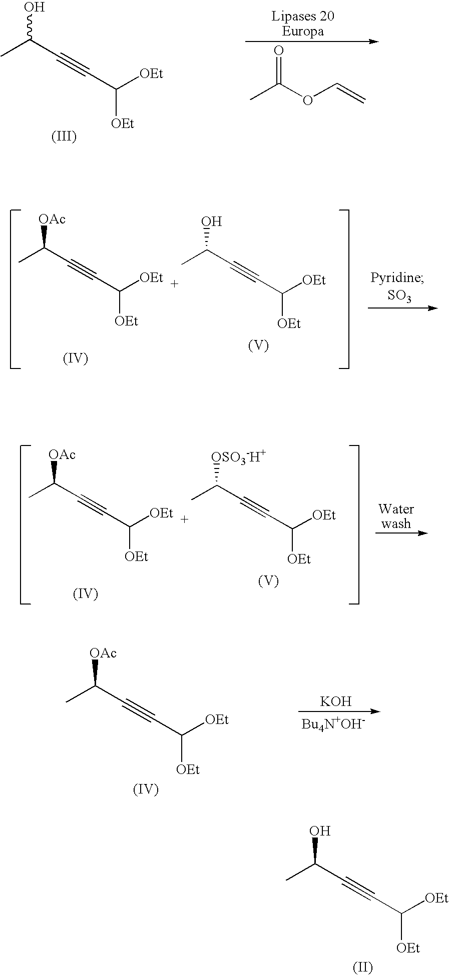

For resolution, 8 g (III), 9.7 g of vinyl acetate, and 0.5 g of Lipase 20 (Europa) were mixed together in 47 ml of MeCN. The reaction was agitated at 25° C. for 22 h. The conversion was 49% by GC analysis with the following method via GC with FID detection:

| | |
|---|---|
| Column: | β-dex 110 (Supelco), 30 m × 0.25 mm × 0.25μ |
| Carrier gas: | Helium 1.5 ml/min |
| Inlet: | 180° C. |
| Split ratio: | 1:100 |
| Oven temperature: | isothermal at 95° C. |
| Retention time: | |
| (R)-(III) | 50.8 min |
| (S)-(III) | 51.8 min |
| (R)-(IV) | 66.5 min |
| (S)-(IV) | 64.4 min |

The products were (R)-(IV) in 99.8% ee, and (S)-(V) in 98.0% ee.

After enzyme removal by filtration, 4.5 ml 1 M DMF solution of SO₃.Pyr was added to the mixture. The reaction was agitated at 35° C. for 4 h to convert (S)-(V) completely to (S)-(VI). After washing with water, only (R)-(IV) remained in the organic phase.

In deacetylation, the TBME solution of acetate (R)-(IV) was mixed with 15 ml 20% KOH and 1.2 g Bu₄N⁺OH⁻. The reaction was agitated at 25° C. for 20 h to completion. After aqueous work up and solvent evaporation, 2.8 g oil was obtained. The identity of (R)-(II) was confirmed with ¹H NMR and GC. The ee was 97% for R enantiomer.

### Example 4

### Screen for Enzymes to Resolve (III)

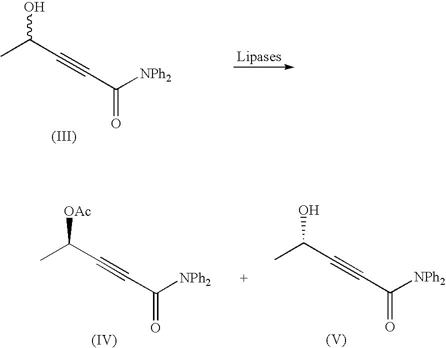

In the screen, each reaction contained 10 mg of (III), 17 mg of vinyl acetate, 10 mg of a lipase, and 1 ml TBME or MeCN. The reactions were agitated at 25° C. After 24 h, the reactions were analyzed for (III) and the corresponding acetate (IV) via HPLC with UV detection at 260 nm:

| | |
|---|---|
| Column: | Chiracel OJ-H, 0.46 × 25 cm, Diacel Chemical |
| | Industries, Ltd. |
| Mobile phase: | 40% iPrOH in Hexanes |
| Flow: | 1 ml/min, isocratic |
| Retention times: | |
| (R)-(III) | 8.2 min |
| (S)-(III) | 6.9 min; |
| (R)-(IV) | 21.7 min |
| (S)-(IV) | 14.3 min |

In total, 55 lipases were screened for the resolution. All exhibited R selectivity in the acylation. Sixteen of the lipases were able to resolve (III) with high selectivity and reached >30% conversion (see Table 6).

TABLE 6
Enzymes Identified to Resolve (III) with Vinyl Acetate

| | | | | ee for 5c | ee for 4c | |
|---|---|---|---|---|---|---|
| Enzyme | Vendor | Solvent | Conversion % | (config.) | (config.) | E |
| Lipase PS | Amano | TBME | 51 | 99.9 (S) | 95.5 (R) | >200 |
| Lipase AK | Amano | MeCN | 30.7 | 44.2 (S) | 99.8 (R) | >200 |
| Lipase PS-C | Amano | MeCN | 47.2 | 89.3 (S) | 99.9 (R) | >200 |
| Lipase PS-D | Amano | MeCN | 50 | 99.9 (S) | 99.9 (R) | >200 |
| Chirazyme L-7 | Biocatalytics | TBME | 41.1 | 69.6 (S) | 99.9 (R) | >200 |
| Chirazyme L-9 | Biocatalytics | TBME | 50.3 | 99.8 (S) | 99.7 (R) | >200 |
| Lipase BC | Biocatalytics | TBME | 43.4 | 76.4 (S) | 99.8 (R) | >200 |
| ICR-107 | Biocatalytics | TBME | 50.6 | 99.8 (S) | 97.4 (R) | >200 |
| ICR-109 | Biocatalytics | MeCN | 32.8 | 48.7 (S) | 99.8 (R) | >200 |
| Lipase 20 | Europa | MeCN | 45.5 | 0.859 (S) | 98.4 (R) | >200 |
| Lipase 4 | Europa | TBME | 51.2 | 99.9 (S) | 95 (R) | >200 |
| Lipase 21 | Europa | MeCN | 41.7 | 71.3 (S) | 99.9 (R) | >200 |
| High Lipase | Sci. Protein labs | TBME | 40.8 | 68.8 (S) | 99.8 (R) | >200 |
| PEC | | | | | | |
| Lipase CE | Sci. Protein labs | TBME | 49.7 | 98.8 (S) | 99.9 (R) | >200 |
| Fungal esterase | Interspex | TBME | 47.7 | 91.1 (S) | 99.9 (R) | >200 |
| ISC-03-FE1 | | | | | | |
| Penicillium | Julich | MeCN | 34.8 | 53.3 (S) | 99.8 (R) | >200 |
| Acylase | | | | | | |

### Example 5

### Deacetylation of (IV) by Methanolysis

Compound (IV) is unstable under basic conditions. General ester hydrolysis with a base such as KOH caused complete degradation. Alcoholysis of (IV) was tested in MeOH, or EtOH with several bases including NaOH, KOH, K₂CO₃ or NaHCO₃. Only NaHCO₃ offered (III) as the major product. Further optimization was conducted in MeOH and EtOH at two temperatures.

In each test, 10 mg of (IV) was added to 1 ml alcohol containing 100 mg NaHCO₃. The reactions were sampled periodically to monitor the progress. Yields were estimated by reverse phase HPLC (general analytical method):

Column: Synergy Polar-RP, 74×4.6 mm, 4μ

Mobile phases:

A: 5% MeCN in water 5 mM HCOOH

B: 95% MeCN in water 5 mM HCOOH

Flow:

| Time | Flow | | | |
|---|---|---|---|---|
| (min) | (1 ml/min) | A % | B % | Curve |
| 0 | 1 | 65 | 35 | n/a |
| 14 | 1 | 50 | 30 | 6 |
| 18 | 1 | 10 | 90 | 6 |
| 20 | 1 | 65 | 35 | 6 |

Detection: 260 nm

Methanolysis with NaHCO₃ at 10° C. turned out to be suitable to deacetylate (I) (see Table 7).

TABLE 7
Alcoholysis of (IV) with NaHCO₃ as the Base

| | | | Time to | |
|---|---|---|---|---|
| | | Temperature | finish | Yield |
| Run | Solvent | (° C.) | (h) | (%) |
| 1 | MeOH | 25 | 2 | 91 |
| 2 | EtOH | 25 | >24 | 81 |
| 3 | MeOH | 10 | 4 | 95 |
| 4 | EtOH | 10 | 22 | 91 |

### Example 7

### Multi-Gram Scale, One-Pot Preparative Resolution of (III) with Lipase PS-D (Amano)

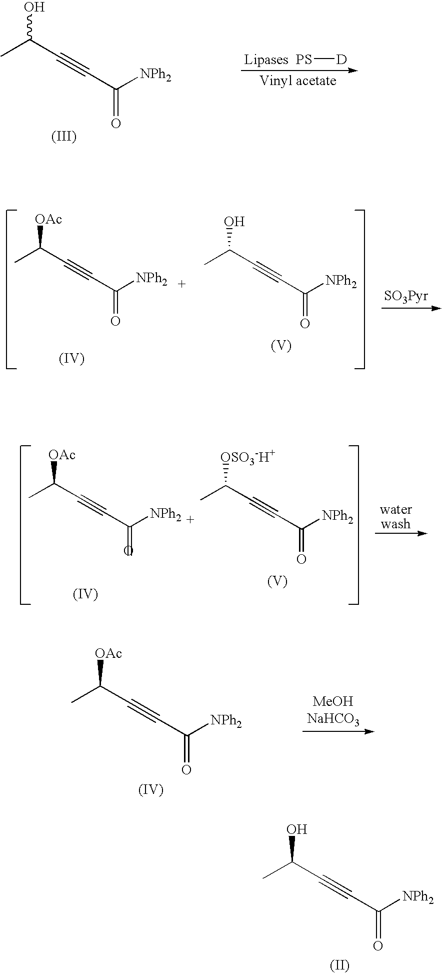

In the resolution, 12 g racemic (III) was mixed with 7.8 g vinyl acetate, and 0.8 g lipase PS-D in 100 ml MeCN. The reaction was agitated at 25° C. The progress of the reaction was monitored by analysis for (III) and the corresponding acetates (IV) via HPLC with UV detection at 260 nm:

| | |
|---|---|
| Column: | Chiracel OJ-H, 0.46 × 25 cm, Diacel Chemical |
| | Industries, Ltd |
| Mobile phase: | 40% iPrOH in Hexanes |
| Flow: | 1 ml/min, isocratic |
| Retention times: | |
| (R)-(III) | 8.2 min |
| (S)-(III) | 6.9 min; |
| (R)-(IV) | 21.7 min |
| (S)-(IV) | 14.3 min |

After 48 h, the conversion reached 47.7%, giving (R)-(IV) in >99% ee and (S)-(V) in 96.7% ee. The enzyme was removed by filtration. The solvent MeCN was evaporated and the solution was reconstituted in 100 ml TBME. In sulfonation, 5.6 g SO₃.Pyr was added and the reaction was agitated at 35° C. After 2 h, all (S)-(V) was converted to the corresponding sulfonate (VI), which was readily removed by washing with water.

For deacetylation, TBME was removed and the solution was reconstituted in 100 ml MeOH. The solution was chilled to 5° C. before adding 7.6 g NaHCO₃ to initiate the reaction. After agitation at 10° C. for 6.5 h, the conversion of (R)-(IV) reached 97%. The reaction was quenched by adding 100 ml EtOAc and removal of NaHCO₃ by filtration. After aqueous workup, 6.2 g of (R)-(II) was obtained. The product (R)-(II) was 94.5% in purity with 98.6% ee.

### Example 8

### The Inversion of Alcohol (II) via Sulfonate (VI)

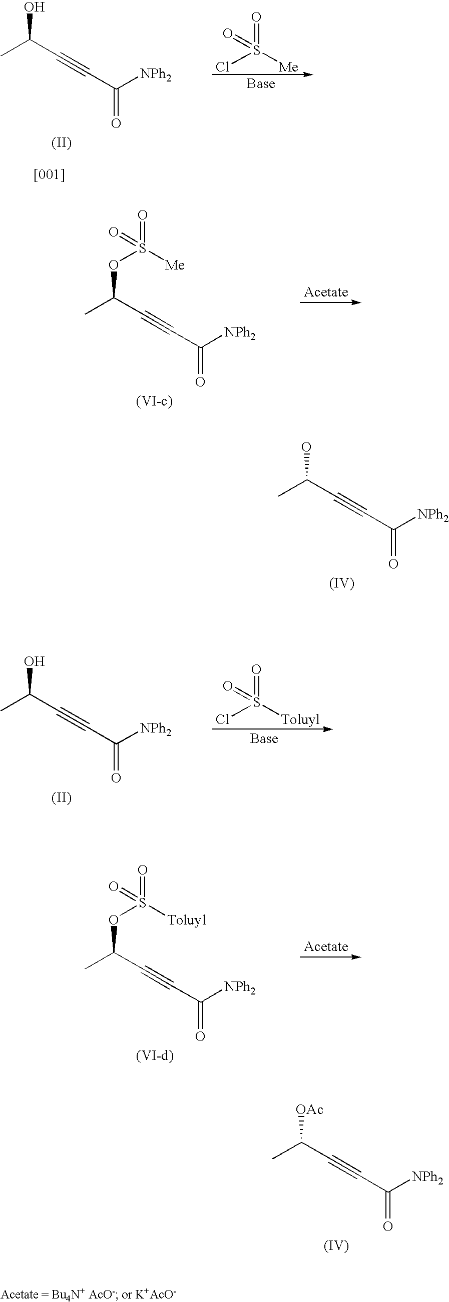

The inversion of the alcohol allows the conversion of the (S)-(II) to (R)-(II). When resolution and the inversion are combined, the theoretical yield of (R)-(II) will be 100%.

The strategy of inversion includes the sulfonation of a chiral alcohol to give a sulfonate (compound (VI-c), or (VI-d), followed by a displacement by an acetate. The product after displacement is acetate (IV) of the opposite enantiomer.

This reaction was carried out in a number of ways. The sulfonate could either be mesylate or tosylate. The bases used in the sulfonation were either Et₃N or DABCO. For displacement, the conditions were dependent on the acetates in use. For Bu₄N⁺AcO⁻, the displacement was carried out in a hydrophobic solvent such as toluene; for K⁺ AcO⁻, the displacement was either in a polar solvent such as DMSO, or in a multiphasic system with a phase transfer catalyst such as Bu₄N⁺HSO₄⁻.

To prepare mesylate (R)-(VI-c), 1.4 g (R)-(II) was dissolved in 30 ml THF. The solution was chilled to 0° C. To this solution, 0.35 g of DABCO (1,4-diazabicyclo[2,2,2]octan) was dissolved, followed by addition of 0.71 g mesyl chloride over 10 min. After agitating at 0° C. for 30 min, the conversion to (VI-c) was complete. The reaction was quenched by adding 30 ml 5% sulfuric acid. After aqueous work up, THF was evaporated and the solution was reconstituted in 20 ml toluene for displacement reaction. In the displacement, 1.6 g K⁺ AcO⁻, 185 mg of Bu₄N⁺HSO₄⁻, and 50 μl of water were added to the toluene solution. This mixture was agitated at 40° C. In 20 h, all (VI-c) was converted, with (IV) as the major product. After aqueous work up and solvent removal, 1.5 g (IV) was obtained. It had 98% ee for S enantiomer as determined by HPLC with UV detection at 260 nm:

| | |
|---|---|
| Column: | Chiracel OJ-H, 0.46 × 25 cm, Diacel Chemical |
| | Industries, Ltd |
| Mobile phase: | 40% iPrOH in Hexanes |
| Flow: | 1 ml/min, isocratic |
| Retention times: | |
| (R)-(III) | 8.2 min |
| (S)-(III) | 6.9 min; |
| (R)-(IV) | 21.7 min |
| (S)-(IV) | 14.3 min |

To prepare tosylate (R)-(VI-d), 23 g of (R)-(II) was dissolved in 180 ml of toluene. The solution was chilled to 0° C. before 13.6 g DABCO and 0.52 g DMAP were added. To this mixture, a tosyl chloride solution (21.5 g in 40 ml MeCN) was added over 30 min. The reaction was agitated for an additional 30 min. to complete the conversion from (R)-(II) to (R)-(VI-d). The reaction was quenched by adding 150 ml 5% sulfuric acid. After aqueous work up and solvent removal, 36.4 g of an oil was obtained. The identity of the product (IV) was confirmed with reverse phase HPLC and ¹H NMR.

The displacement of (R)-(VI-d) with Bu₄N⁺AcO⁻ was conducted in toluene. (R)-(VI-d) (36 g) was dissolved in 150 ml toluene. The reaction was chilled to 10° C. before Bu₄N⁺AcO⁻ (39.2 g in 80 ml MeCN) was added over 30 min. After agitating for 7 h at 10° C., all (VI-d) was converted, mostly to (IV). After work up, 21.5 g (IV) was obtained. The identity was confirmed with HPLC and ¹H NMR. The ee was determined to be 98% for the (S)-enantiomer.

In the displacement of (R)-(VI-d) with K⁺ AcO⁻ in DMSO, 1 g of (R)-(VI-d) and 0.7 g of the acetate was mixed in 5 ml solvent. The reaction was agitated at 25° C. After 40 h, the conversion reached 97%. To work up, 20 ml of EtOAc was added to the reaction mixture. The solution was washed by 5% sulfuric acid, 5% NaHCO₃, and brine. After solvent removal, 0.82 g of (IV) was obtained. The identity was confirmed with HPLC and ¹H NMR. The ee was determined to be 98% for the (S)-enantiomer.

In the displacement of (R)-(VI-d) with K⁺ AcO⁻ by phase transfer catalysis, 11 g of (R)-(VI-d) was mixed with 7.7 g K⁺ AcO⁻, 1.8 g of Bu₄N⁺HSO₄⁻, 1 ml water, and 0.66 ml acetic acid in 66 ml toluene. The reaction was agitated at 55° C. After 22 h, the conversion was complete based on reverse phase HPLC (general analytical method):

Column: Synergy Polar-RP, 74×4.6 mm, 4μ

Mobile phases:
A: 5% MeCN in water 5 mM HCOOH
B: 95% MeCN in water 5 mM HCOOH

Flow:

| Time | Flow | | | |
|---|---|---|---|---|
| (min) | (1 ml/min) | A % | B % | Curve |
| 0 | 1 | 65 | 35 | n/a |
| 14 | 1 | 50 | 30 | 6 |
| 18 | 1 | 10 | 90 | 6 |
| 20 | 1 | 65 | 35 | 6 |

Detection: 260 nm

The reaction was quenched with 45 ml 8% sulfuric acid. After aqueous work up and solvent removal, 8.4 g of (IV) was obtained. The identity was confirmed with HPLC and ¹H NMR. The ee was determined to be 94% for the (S)-enantiomer.

### Example 9

### The Enzymatic Hydrolysis of (IV)

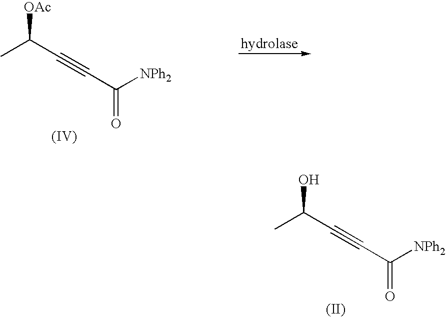

The deacetylation of (R)-(IV) by enzyme hydrolysis offers several advantages: the reaction condition is mild and the hydrolysis is efficient, so that the degradation of (IV) is minimized. More importantly, enzyme hydrolysis is R selective for (IV), offering additional enantioselectivity for making the product.

The identification of the enzyme started from screening 53 commercially available enzymes for the hydrolysis of (R)-(IV). Typically, the reaction mixture in the screen included 20 mg of (R)-(IV) in 0.2 ml toluene, 20 mg of an enzyme, and 0.8 ml of 0.2 M phosphate buffer, pH 7.0. The reaction was agitated at 35° C. for 1.5 h. The conversion was determined by reverse phase HPLC. There were 13 reactions that showed ≧30% conversion (see Table 8). CALB L was picked for further testing.

In the test, the reaction included 0.2 g CALB L, 150 mg of racemic (IV) in a mixture of toluene: water (0.6:6). After agitation at 40° C. for 1.5 h, the conversion reached 49.2%. The products were (R)-(II) in 96.2% ee, and (S)-(IV) in 99.5% ee. The enantiomeric ratio (E) was 1482 for (R)-(IV).

TABLE 8
Enzymes Identified in Hydrolyzing (IV)

| Hydrolyzing Enzyme | Vendor | Conversion % |
|---|---|---|
| LPS | Amano | 98 |
| Lipoprotein lipase | Amano | 98 |
| 200S | | |
| Lipase PS-C | Amano | 42 |
| Chirazyme L6 | Biocatalytics | 52 |
| Lipase BC | Biocatalytics | 30 |
| ICR-107 | Biocatalytics | 31 |
| Lipase 4 | Europa | 54 |
| Lipase 3 | Europa | 97 |
| Lipase B | Novozyme | 56.1 |
| LPL-311 TypeA | Toyobo | 70.5 |
| LPL-701 | Toyobo | 49 |
| Cholesterol esterase | Amano | 39 |
| CALB L | Novozyme | 44 |

CALB L hydrolysis was optimized in terms of pH (6-9), temperature (25° C.-45° C., and the amount of toluene (2× to 10×)).

### Example 10

### The Preparation of (R)-(III) from its Racemate by Resolution/Inversion Strategy

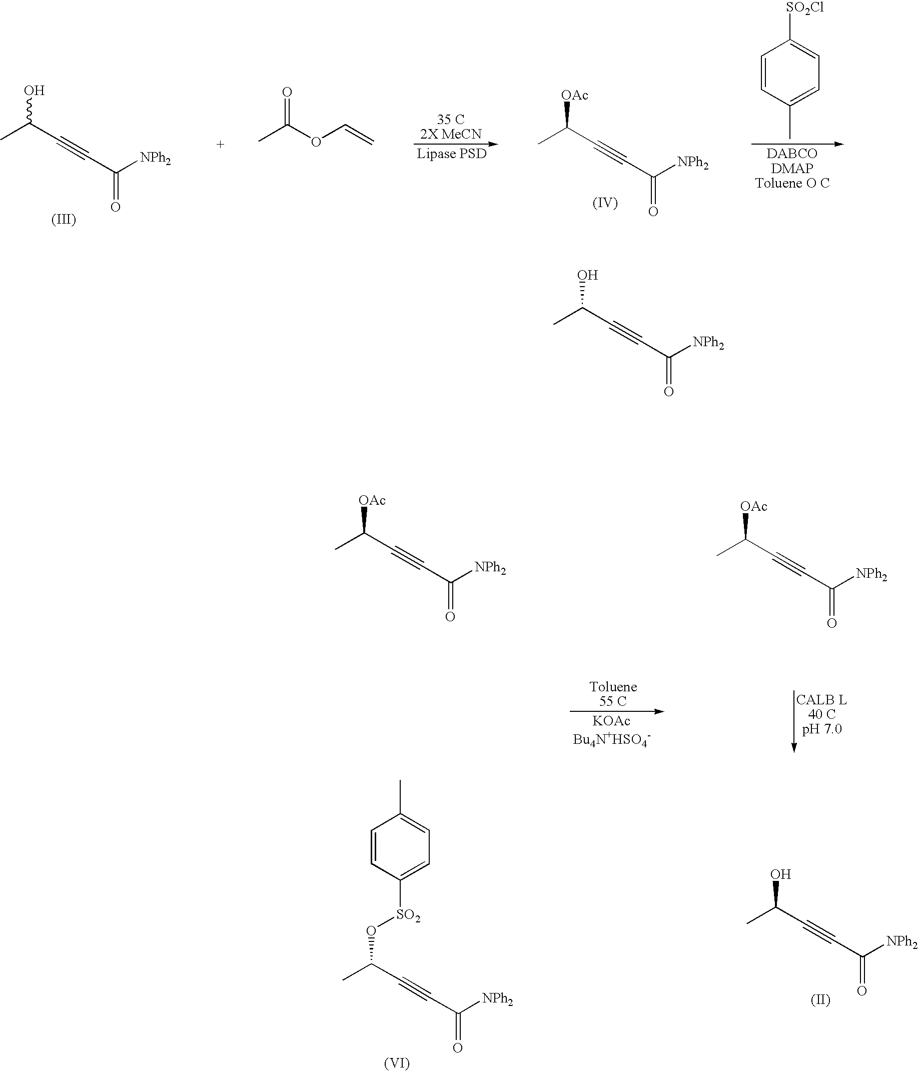

The resolution was carried out by mixing 50 g (III) with 65 g vinyl acetate, and 3 g of lipase PS-D in 100 ml MeCN. The reaction was agitated at 35° C. After 30 h, the conversion was 48.8%. The products included (R)-(IV) in 99.8% ee, and (S)-(V) in 95.1% ee. After removal of solvent and enzyme, the solution was reconstituted in 300 ml toluene for tosylation.

In tosylation, the toluene solution was chilled to 0° C. followed by adding TsCl solution (21.6 g in 30 ml of MeCN). To this mixture, a solution of DABCO and DMAP (13.7 g and 0.6 g, respectively, in 60 ml MeCN) was added over 30 min. The reaction was agitated at 0° C. for an additional 30 min to complete (>99% conversion). The reaction was quenched by adding 200 ml 8% H₂SO₄. After the removal of the aqueous phase, the organic layer was washed with 200 ml 8% NaHCO₃, and 200 ml brine.

The displacement of tosylate (S)-(VI) with K⁺ AcO⁻ was conducted under phase transfer conditions. To the solution from the previous step, 27.7 g K⁺ AcO⁻, 6.4 g catalyst Bu₄N⁺AcO⁻, 3.3 ml of AcOH, and 3.3 ml of water were added. The reaction was agitated at 55° C. In 24 h, the conversion of (VI) reached 93%. The reaction was quenched by adding 200 ml 8% H₂SO₄. After the removal of the aqueous phase, the organic layer was washed with 200 ml 8% NaHCO₃. The solution was concentrated to a final volume of 150 ml by distillation.

In the deacetylation step, 250 ml of 0.1 M phosphate buffer (pH 7.0) was added to the solution from the last step. CALB L (10 g) was charged to the solution to initiate the hydrolysis. The reaction mixture was vigorously agitated at 35° C. The pH was maintained at 7.0 by titrating 1 M NaOH with a pH stat. In 20 h the conversion reached 96%, giving (II) as the major product. To work up, 200 ml EtOAc was added to the mixture. The solution was filtered and then washed with 200 ml 8% H₂SO₄, 200 ml 8% NaHCO₃, and 200 ml 30% brine.

The product (II) was purified by crystallization in a 700 ml mixture of heptane and EtOAc (6:1). In total, 35.0 g crystalline was obtained. The purity of the (R)-(II) product was 99% and ee was 99.6%.

### Example 11

### A Process for Preparing (R)-(I) by Lipase Catalyzed Enantioselective Coupling

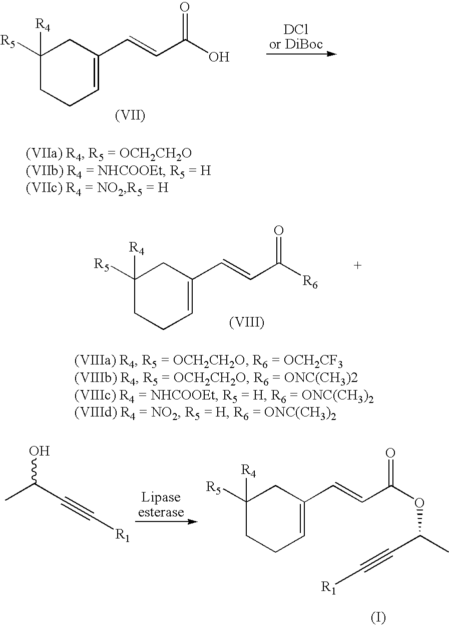

Coupling of acid (VII) selectively with (R)-(II) from its racemic mixture provides a more efficient access to the critical intermediate (R)-(I) by saving one step. Lipase usually catalyzes such coupling through an active ester (VIII).

In the screen for lipases, the substrate was 2,2,2-Trifuoroethanol ester (VIIIa). Compound (VIIIa) was prepared by CDI (carbonyl diimidazole) mediated esterification of (VIIa) with 2,2,2-Trifuoroethanol.

After dissolving 25 g CDI in 100 ml THF, 29.4 g (VIIa) was added. The reaction mixture was agitated at 25° C. for 1 h before adding 19.3 g 2,2,2-Trifuoroethanol and 1.4 ml of a 1 M THF solution of LiOEt. The reaction was agitated for an additional 20 h at 25° C. to completion. The reaction was quenched by adding 50 ml saturated NH₄Cl. The aqueous phase was discarded and THF was replaced by 250 ml TBME. After aqueous work up and solvent removal, 42.4 g of (VIIIa) was obtained. The purity was determined to be 95%.

The screen for lipases was carried out by testing 53 lipases or esterases in the coupling of (VIIIa) with (IIIa). Each reaction contained 8 mg of (VIIIa), 10 mg of (IIIa), 10 mg of a lipase, and 1 ml of TBME or MeCN. The reactions were agitated at 25° C. for 18 h. The reaction was analyzed first by TLC. For those reactions that gave product (I), the ester was separated by TLC for ee determination. In ee deterimation, (I) was first hydrolyzed by 1 M NaOH containing 10% Bu₄N⁺HSO₄⁻ for 12 h at 25° C. to give (IIIa) and (VIIa). The ee of (IIIa) product was determined via GC with FID detection.

| | |
|---|---|
| Column: | β-dex 110 (Supelco), 30 m × 0.25 mm × 0.25μ |
| Carrier gas: | Helium 1 ml/min |
| Inlet: | 180° C. |
| Split ratio: | 1:100 |
| Oven temperature: | isothermal at 100° C. |
| Retention time: | (R)-(IIIa) 30.8 min |
| | (S)-(IIIa) 31.9 min |

Three lipases/esterases were found to catalyze the coupling reaction in TBME (see Table 9). All of them were R selective. Chirazyme L 9 exhibited the highest activity.

TABLE 9
The enzymes identified in the coupling of (VIIIa) and (IIIa)

| Coupling | | | | ee |
|---|---|---|---|---|
| Enzyme | Vendor | Solvent | Conversion % | (Config) |
| Chirazyme L-9 | Biocatalytics | TBME | 100 | 89% (R) |
| EnzecoEsterase/ | EDC | TBME | 50 | 74% (R) |
| Lipase | | | | |
| Cholesterol | Amano | TBME | 32 | 67% (R) |
| esterase | | | | |

### Example 12

### Lipase-Catalyzed Coupling of Oxime Ester (VIII) with (R)-(III) from its Racemic Mixture and the Transformation of Product (1) for ee Determination

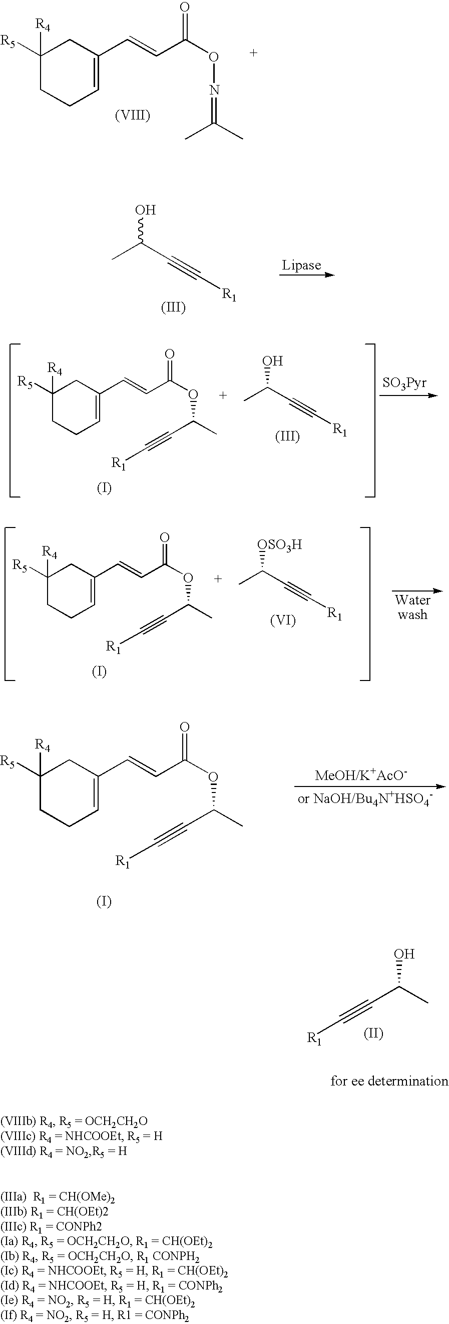

Several active esters of (VII) were compared for their efficiency in Chirazyme L-9 catalyzed coupling with (III). These esters included vinyl, isopropenyl, 1-ethoxyvinyl and oxime esters. All the vinyl esters were unstable in TBME. The oxime ester (VIIIb) turned out to be the best. It was stable and the reaction rate was 1.5 times faster than when (VIIIa) was the substrate. This coupling was also carried out in several solvents such as MeCN, acetone, 4-methyl-2-pentanone, toluene, t-BuOAc, t-amyl alcohol, and THF. In 4-Me-2-pentanone, and t-BuOAc, the reaction rates were comparable to that in TBME.

Chirazyme L-9 was tested in the coupling of oxime esters including (VIIIb), (VIIIc) and (VIIId) with (IIIb) and (IIIc).

The oxime ester was prepared by DiBoc (Di-tert-Butyl carbonate) mediated esterification. In preparation of (VIIIb), 30.1 g of (VIIa) was mixed with 12.6 g of acetone oxime, 14.7 g of pyridine, and 2.6 g of DMAP in 280 ml THF. The mixture was agitated at 25° C. The acid activation reagent (t-BuOOC)₂O (12.6 g in 20 ml THF) was then added over 10 min. After 24 h at 25° C., the reaction was complete with (VIIIb) as the only product. The solvent was removed and the solution was reconstituted in 600 ml EtOAc. After aqueous work up and solvent removal, 30.6 g (VIIIb) was obtained. Oxime ester (VIIIc) and (VIIId) were prepared similarly.

In the coupling of (VIIIb) with (IIIc), 100 mg of (VIIIb), 400 mg of (IIIc), and 100 mg of Chirazyme L-9 were mixed in 6 ml TBME. The reaction was agitated at 35° C. Samples were taken and analyzed by reverse phase HPLC to monitor the progress:

Column: Synergy Polar-RP, 74×4.6 mm, 4μ

Mobile phases:
A: 5% MeCN in water 5 mM HCOOH
B: 95% MeCN in water 5 mM HCOOH

Flow:

| Time | Flow | | | |
|---|---|---|---|---|
| (min) | (1 ml/min) | A % | B % | Curve |
| 0 | 1 | 65 | 35 | n/a |
| 14 | 1 | 50 | 30 | 6 |
| 18 | 1 | 10 | 90 | 6 |
| 20 | 1 | 65 | 35 | 6 |

Detection: 260 nm

There were two products in the reaction. The major product was ester (Ib), and the minor product was the corresponding acid (VIIa) from hydrolysis. When conversion reached >90%, 10 ml EtOAc was added to the reaction mixture. Chirazyme L-9 was removed and then the reaction mixture was washed with 20 ml 5% NaHCO₃, and 20 ml brine. The solution was dried over Na₂SO₄ before sulfonation. To remove the unreacted alcohol (IIIc), 0.32 g Pyr.SO3 and 2 ml DMF was added to the mixture and the solution was agitated at 35° C. After 12 h, alcohol (IIIc) was completely converted to sulfonate (VIb), which was removed by washing with water. After solvent removal, 127 mg of an oil was obtained, whose identity was proven to be (Ib) by ¹H NMR.

To determine the ee of the product (Ib), 20 mg of the product was added to 1 ml pre-chilled MeOH containing 1 g of KHCO₃. After agitating for 16 h at 0° C., >99% of (Ib) was converted to the corresponding methyl ester and (IIc). After salt removal and solvent evaporation, (IIc) was purified by TLC. Its ee was determined to be 98.6% for (R)-(IIc) by HPLC with UV detection at 260 nm.

Column: Chiracel OJ-H, 0.46×25 cm, Diacel Chemical Industries, Ltd

Mobile phase: 40% iPrOH in Hexanes

Flow: 1 ml/min, isocratic

The couplings of other substrates were carried out similarly. The results were summarized in Table 10. In all cases, the conversion was complete, giving the ester product (I) as the major product and corresponding acid (VII) as a minor product. The ee for these products were all >98% for (R)-enantiomer.

TABLE 10
Summary of the Results of Chirazyme L-9 Catalyzed Coupling

| | | | ee % |
|---|---|---|---|
| Ester | Conversion % | Yield % | (Config.) |
| 3a | 99 | 60 | >99 (R) |
| 3b | 100 | 74 | 98.6 (R) |
| 3c | 91 | 59 | >99 (R) |
| 3d | 100 | 40 | 98.5 (R) |
| 3e | 95 | 58 | 98.9 (R) |
| 3f | 100 | 71 | >99 (R) |

### Example 13

### Multi-gram Coupling of (VIIIb) with (IIIb) by Chirazyme L-9

The reaction was carried out by the strategy outlined in Example 12. In the coupling, 3.98 g of (VIIIb), 6.45 g (IIIb), 1.5 g chirazyme L9 were mixed in 45 ml dry TBME. The reaction was agitated at 35° C. After 44 h, the conversion reached 98.2%, giving approximately 80% product (Ia), and 18% of acid (VIIa). The enzyme was removed by filtration. For removal of the remaining (IIIb), 6.6 g of SO₃.Pyr, and 10 ml methylene chloride were added. Compound (IIIb) was completely converted to sulfonate (Va) after agitation for 14 h at 35° C. The organic phase was washed with 200 ml water, and 275 ml of 5% K₂CO₃. After drying and solvent removal, 4.91 g of (Ia) (92.4% in purity) was obtained, representing 83% yield. The ee of the product was determined to be >99% for R enantiomer.

### Example 14

### Multigram Coupling of (VIIId) with (IIIc) by Chirazyme L-9

The coupling was carried out by the scheme outlined in Example 12. In the coupling, 2.52 g of (VIIId), 6.63 g of (IIIc), and 1.2 g of chirazyme L-9 were mixed in 75 ml of dry TBME. The reaction was agitated at 35° C. After 96 h, the conversion reached 97.5%, giving approximately 75% of the product (Id), and 25% of the corresponding acid (VIIc). To remove the remaining (IIIc), 50 ml EtOAc and 4.3 g of SO₃.Pyr in 5 ml DMF were added to the mixture. The agitation was continued for 2 h to complete the sulfonation. The insoluble was then removed by filtration. The organic solution was washed with 5% acetic acid, 8% KHCO₃, and brine, 150 ml each. After concentration, the crude oil (4.1 g) was purified over a silica gel column. It gave 3.15 g product (If) in >99% purity. NMR analysis indicated that the product was a mixture of two diastereomers. The ee with the respect of (IIIc) moiety was determined to be >99% for R enantiomer.

### Example 15

### Multigram Coupling of (VIIId) with (IIIb) by Chirazyme L-9

The coupling was carried out by the scheme outlined in Example 12. In the coupling, 2.52 g of (VIIId), 4.31 g of (IIIb), and 1.2 g of chirazyme L-9 were mixed in 75 ml of dry TBME. The reaction was agitated at 35° C. After 96 h, the conversion reached 95.4%, giving approximately 70% of the product (Ie), and 30% of the corresponding acid (VIIc). To remove the remaining (IIIb), 50 ml iPrOAc and 4.3 g of SO₃.Pyr in 5 ml DMF were added to the mixture. The agitation was continued for 2 h to complete the sulfonation. The insoluble was then removed by filtration. The organic solution was washed with 5% acetic acid, 8% KHCO₃, and brine, 150 ml each. After concentration, the crude oil (2.6 g) was purified over a silica gel column. It gave 2.15 g product (Ie) in >99% purity. NMR analysis indicated that the product was a mixture of two diastereomers. The ee with the respect of (IIIb) moiety was determined to be 98.1% for R enantiomer.

### Example 16

### Multigram Coupling of (VIIIb) with (IIIc) by Chirazyme L-9

The coupling was carried out by the scheme outlined in Example 12. In the coupling, 2.65 g of (VIIIb), 6.63 g of (IIIc), and 1.2 g of chirazyme L-9 were mixed in 75 ml of dry TBME. The reaction was agitated at 35° C. After 21 h, the conversion reached 97.8%, giving approximately 83% of the product (Ib), and 17% of the corresponding acid (VIIa). To remove the remaining (IIIc), 50 ml EtOAc and 4.5 g of SO₃.Pyr in 5 ml DMF were added to the mixture. The agitation was continued for 2 h to complete the sulfonation. The insoluble was then removed by filtration through celite. The organic solution was washed with 5% acetic acid, 8% KHCO₃, and brine, 150 ml each. After concentration, the crude oil (3.8 g) was purified over a silica gel column. It gave 3.20 g product (Ib) in >99% purity. The ee was determined to be >99% for R enantiomer.

While the present invention has been described in conjunction with the specific embodiments set forth above, many alternatives, modifications and variations thereof will be apparent to those of ordinary skill in the art. All such alternatives, modifications, and variations are intended to fall within the spirit and scope of the present invention.

What is claimed is:

1. A process for preparing a compound of formula (I): 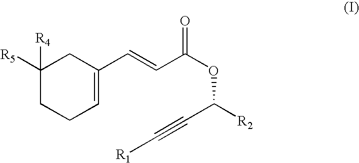
from a compound of formula (II): 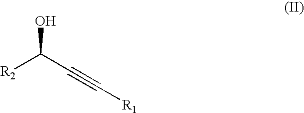
said process comprising:
(a) reacting a compound of formula (III): 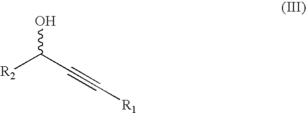
with an acetate in the presence of a resolving enzyme to yield compounds of formulae (IV) and (V): 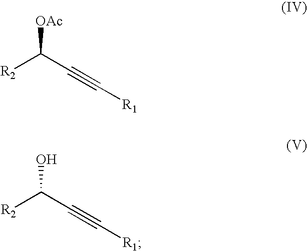
(b) sulfonating the compound of formula (M to yield a sulfonate compound of formula (VI): 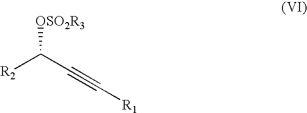
said sulfonate compound of formula (VI) being either removed by washing with water or converted to acetate compound of formula (IV) by displacement of sulfonate group to acetate group;
(c) converting the compound of formula (IV) to the compound of formula (II); and,
(d) esterifying a compound of formula (VII): 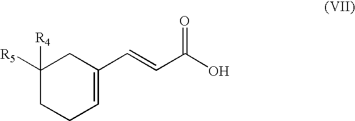
with the compound of formula (II) to yield the compound of formula (I),
wherein R₁ and R₂ are each independently selected from the group consisting of hydrogen, halogen, alkyl, haloalkyl, alkoxy, mono- and di-alkoxyalkyl, alkenyl, alkynyl, mono- and di-alkylamino, mono- and di-arylamino, (aryl)alkylamino, (alkyl)arylamino, amido, mono- and di-alkylamido, and mono- and di-arylamido groups,
R₃ is selected from the group consisting of alkyl, aryl, arylalkyl, and heteroaryl groups,
R₄ and R₅ are each independently selected from the group consisting of H, hydroxyl, amino, nitro, amido, halogen, alkyl, alkenyl, alkoxy, mono- and di-alkoxyalkyl-, alkoxyalkyl, halo(C₁-C₆ alkyl)-, dihaloalkyl-, trihaloalkyl-, cycloalkyl, cycloalkyl-alkyl-, aryl, alkyl-aryl, aryl-alkyl-, thioalkyl, alkyl-thioalkyl, alkenyl, hydroxyl-alkyl-, aminoalkyl-, —C(O)OR₇, —C(O)NR₈R₉, -alkyl-C(O)NR₈R₉, —NR₁₀R₁₁, and N₁₀R₁₁-alkyl, or R₄ and R₅ together with the carbon to which they are attached, form a heteroaryl or heterocyclic group of 5 to 10 atoms comprised of hydrogen atoms, 1 to 9 carbon atoms and 1 to 4 heteroatoms independently selected from the group consisting of N, O, and S, wherein a ring nitrogen can form an N-oxide or a quaternary group with a (C₁-C₄)alkyl group;
R₇, R₈ and R₉ are each independently selected from the group consisting of H, (C₁-C₆)alkyl, phenyl, and benzyl; and
R₁₀ and R₁₁ are each independently selected from the group consisting of H and (C₁-C₆)alkyl.

2. The process of claim 1, wherein R₁ is selected from the group consisting of mono- and di-alkoxyalkyl and N,N-diarylamido groups.

3. The process of claim 2, wherein R₁ is selected from the group consisting of dimethoxymethyl, diethoxymethyl, and N,N-diphenylamido groups.

4. The process of claim 1, wherein R₂ is methyl.

5. The process of claim 1, wherein the compound of formula (V) is sulfonated with a compound selected from the group consisting of SO₃.Pyr and R₃SO₂X, wherein R₃ is selected from the group consisting of alkyl, aryl, arylalkyl, and heteroaryl groups, and X is halogen.

6. The process of claim 1, wherein R₃ is selected from the group consisting of alkyl and aryalkyl groups.

7. The process of claim 1, wherein R₃ is selected from the group consisting of methyl and toluyl groups.

8. The process of claim 1, wherein the compound of formula (V) is sulfonated in the presence of a base.

9. The process of claim 8, wherein the base is selected from the group consisting of triethylamine, 1,4-diazabicyclo[2,2,2]octane, and DMAP.

10. The process of claim 1, wherein at least a portion of the resolving enzyme is optionally removed prior to sulfonation.

11. The process of claim 1, wherein the compound of formula (VI) is removed from the reaction mixture by washing with water.

12. The process of claim 1, wherein the compound of formula (VI) is subjected to chiral inversion by reacting said compound with a salt of an organic acid to yield a compound of formula (IV).

13. The process of claim 12, wherein the organic acid is acetic acid.

14. The process of claim 12, wherein the chiral inversion is conducted in a multiphasic system in the presence of a phase transfer catalyst.

15. The process of claim 12, wherein the chiral inversion is conducted in a monophasic system in the presence of a nucleophile.

16. The process according to claim 15, wherein the nucleophile is an acetate salt.

17. The process of claim 16, wherein the acetate salt is selected from the group consisting of tetrabutyl ammonium acetate and potassium acetate.

18. The process of claim 1, wherein the resolving enzyme is selected from at least one of the group consisting of lipases, proteases, peptidases, amidases, acylases, and esterases.

19. The process of claim 18, wherein the resolving enzyme is a lipase.

20. The process of claim 1, wherein the compound of formula (III) reacts with an acetate in the presence of a solvent.

21. The process of claim 20, wherein the solvent is selected from the group consisting of t-butyl methyl ether and acetonitrile.

22. The process of claim 1, wherein the compound of formula (IV) is converted to the compound of formula (II) by deacetylation.

23. The process of claim 22, wherein the deacetylation is conducted in the presence of a base.

24. The process of claim 23, wherein the base is selected from the group consisting of alkali metal hydroxides, tetraalkylammonium hydroxides, and combinations thereof.

25. The process of claim 24, wherein the base is a mixture comprising potassium hydroxide and tetrabutylammonium hydroxide.

26. The process of claim 1, wherein the compound of formula (IV) is converted to the compound of formula (II) by alcoholysis.

27. The process of claim 26, wherein the alcoholysis is conducted in the presence of an alcohol selected from the group consisting of (C₁-C₆) alkanols.

28. The process of clam 27, wherein the alcoholysis is conducted in the presence of a base.

29. The process of claim 28, wherein the base is selected from the group consisting of alkali metal carbonates.

30. The process of claim 29, wherein the alkali metal carbonate is NaHCO₃ or KHCO₃.

31. The process of claim 1, wherein the acetate is selected from the group consisting of alkyl and alkenyl acetates.

32. The process of claim 31, wherein the alkenyl acetate is vinyl acetate.

33. The process of claim 1, wherein the compound of formula (IV) is converted to the compound of formula (II) by hydrolysis.

34. The process of claim 33, wherein the hydrolysis is enzymatic hydrolysis.

35. The process of claim 34, wherein the enzymatic hydrolysis is conducted with a hydrolase.

36. The process of claim 33, wherein the hydrolysis is conducted in the presence of a solvent.

37. The process of claim 36, wherein the solvent is selected from the group consisting of organic solvents, aqueous solvents, and mixtures thereof.

38. The process of claim 1, wherein said process is a one-pot process.

39. A process for preparing a compound of formula (I): 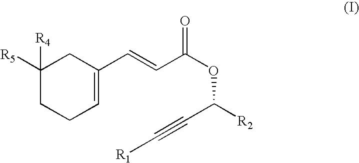
from a compound of formula (VII): 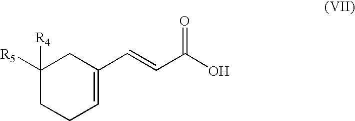
said process comprising:
(a) activating a compound of formula (VII) to yield a compound of formula (VIII): 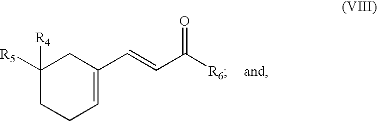
(b) reacting the compound of formula (VIII), in the presence of an enzyme, with a compound of formula (III): 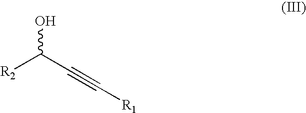
to yield a compound of formula (I),
wherein R₁ and R₂ are each independently selected from the group consisting of hydrogen, halogen, alkyl, haloalkyl, alkoxy, mono- and di-alkoxyalkyl, alkenyl, alkynyl, mono- and di-alkylamino, mono- and di-arylamino, (aryl)alkylamino, (alkyl)arylamino, amido, mono- and di-alkylamido, and mono- and di-arylamido groups;
R₄ and R₅ are each independently selected from the group consisting of H, hydroxyl, amino, nitro, amido, halogen, alkyl, alkenyl, alkoxy, mono- and di-alkoxyalkyl-, alkoxyalkyl, halo(C₁-C₆ alkyl)-, dihaloalkyl-, trihaloalkyl-, cycloalkyl, cycloalkyl-alkyl-, aryl, alkyl-aryl, aryl-alkyl-, thioalkyl, alkyl-thioalkyl, alkenyl, hydroxyl-alkyl-, aminoalkyl-, —C(O)OR₇, —C(O)NR₈R₉, -alkyl-C(O)NR₈R₉, —NR₁₀R₁₁, and N₁₀R₁₁-alkyl, or R₄ and R₅, together with the carbon to which they are attached, form a heteroaryl or heterocyclic group of 5 to 10 atoms comprised of hydrogen atoms, 1 to 9 carbon atoms and 1 to 4 heteroatoms independently selected from the group consisting of N, O, and S, wherein a ring nitrogen can form an N-oxide or a quaternary group with a (C₁-C₄)alkyl group;
R₆ is selected from the group consisting of alkoxy and alkenyloxy, each of which may be unsubstituted or substituted with at least one of halogen atoms and nitro, amino, and (C₁-C₆)alkoxy groups, ONH₂, ONH(CₙH₂ₙ₊₁), ON(CₙH₂ₙ₊₁)(CₙH₂ₙ), ON(CₙH₂ₙ), and ON(CₙH₂ₙ₊₁)₂, wherein n ranges from 1 to 6;
R₇, R₈, and R₉ are each independently selected from the group consisting of H, (C₁-C₆)alkyl, phenyl, and benzyl; and
R₁₀ and R₁₁ are each independently selected from the group consisting of H and (C₁-C₆)alkyl.

40. The process of claim 39, wherein R₁ is selected from the group consisting of alkoxyalkyl and diarylamido groups, and said enzyme in step (b) is Chirazyme L9, Enzeco Esterase/Lipase or Cholesterol esterase.

41. The process of claim 40, wherein R₁ is selected from the group consisting of dimethoxymethyl, diethoxymethyl, and diphenylamido groups.

42. The process of claim 39, wherein R₂ is methyl.

43. The process of claim 39, wherein R₄ and R₅, together with the carbon atom to which they are attached, form a five-membered heterocyclic ring containing two heteroatoms.

44. The process of claim 43, wherein the two heteroatoms are oxygen atoms.

45. The process of claim 39, wherein the compound of formula (VII) is activated by esterification.

46. The process of claim 45, wherein the compound of formula (VII) is esterified with an alcohol.

47. The process of claim 46, wherein the alcohol is selected from the group consisting of (C₁-C₆) alcohols, unsubstituted or substituted with at least one substituent selected from the group consisting of halogen atoms and nitro, amino, and (C₁-C₆)alkoxy groups.

48. The process of claim 47, wherein the alcohol is isopropenyl alcohol.

49. The process of claim 47, wherein the substituted (C₁-C₆) alcohols are halo-substituted alcohols.

50. The process of claim 49, wherein the halo-substituted alcohols are fluorinated alcohols.

51. The process of claim 50, wherein the fluorinated alcohol is 2,2,2-trifluoroethanol.

52. The process of claim 45, wherein the compound of formula (VII) is esterified with an oxime.

53. The process of claim 52, wherein the oxime is of the formula: 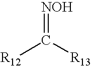
wherein R₁₂ and R₁₃ are each independently selected from the group consisting of a hydrogen atom, alkyl, and alkenyl groups.

54. The process of claim 53, wherein R₁₂ and R₁₃ are methyl.

55. The process of claim 39, wherein the compound of formula (VII) is activated in the presence of a mediator selected from the group consisting of carbonyl diimidazole and di-tert-butyl carbonate.

56. The process of claim 39, wherein the compound of formula (VIII) is reacted with the compound of formula (III) in the presence of a solvent.

57. The process of claim 56, wherein the solvent is selected from the group consisting of acetone, acetonitrile, 4-methyl-2-pentanone, toluene, t-butoxyacetate, t-amyl alcohol, t-butyl methyl ether, and tetrahydrofuran.

58. The process of claim 39, wherein following (b), remaining compound of formula (III) is removed by sulfonation.

59. A process for preparing a compound of formula (II): 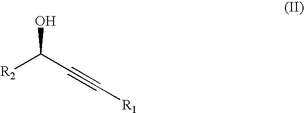
from a compound of formula (III): 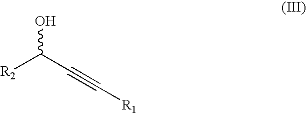
said process comprising:
(a) reacting the compound of formula (III) with an acetate in the presence of a resolving enzyme to yield compounds of formulae (IV) and (V): 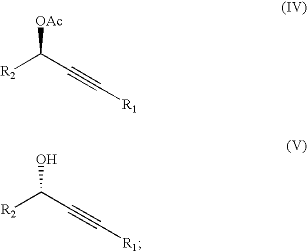
(b) sulfonating the compound of formula (V) to yield a compound of formula (VI): 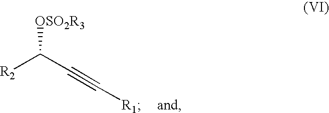
(c) converting the compound of formula (IV) to the compound of formula (II),
wherein R₁ and R₂ are each independently selected from the group consisting of hydrogen, halogen, alkyl, haloalkyl, alkoxy, mono- and di-alkoxyalkyl, alkenyl, alkynyl, mono- and di-alkylamino, mono- and di-arylamino, (aryl)alkylamino, (alkyl)arylamino, amido, mono- and di-alkylamido, and mono- and di-arylamido groups, and
R₃ is selected from the group consisting of hydrogen, alkyl, aryl, arylalkyl, and heteroaryl groups.

60. The process of claim 59, wherein R₁ is selected from the group consisting of mono- and di-alkoxyalkyl and N,N-diarylamido groups.

61. The process of claim 60, wherein R₁ is selected from the group consisting of dimethoxymethyl, diethoxymethyl, and N,N-diphenylamido groups.

62. The process of claim 59, wherein R₂ is methyl.

63. The process of claim 59, wherein the compound of formula (V) is sulfonated with a compound selected from the group consisting of SO₃.Pyr and R₃SO₂X, wherein R₃ is selected from the group consisting of alkyl, aryl, arylalkyl, and heteroaryl groups, and X is halogen.

64. The process of claim 59, wherein R₃ is selected from the group consisting of alkyl and aryalkyl groups.

65. The process of claim 64, wherein R₃ is selected from the group consisting of methyl and toluyl groups.

66. The process of claim 59, wherein the compound of formula (V) is sulfonated in the presence of a base.

67. The process of claim 66, wherein the base is selected from the group consisting of triethylamine and 1,4-diazabicyclo[2,2,2]octane.

68. The process of claim 59, wherein at least a portion of the resolving enzyme is removed prior to sulfonation.

69. The process of claim 59, wherein the compound of formula (VI) is removed from the reaction mixture by washing with water.

70. The process of claim 59, wherein the compound of formula (VI) is subjected to chiral inversion by reacting said compound with a salt of an organic acid to yield a compound of formula (IV).

71. The process of claim 70, wherein the organic acid is acetic acid.

72. The process of claim 70, wherein the chiral inversion is conducted in a multiphasic system in the presence of a phase transfer catalyst.

73. The process of claim 70, wherein the chiral inversion is conducted in a monophasic system in the presence of a nucleophile.

74. The process according to claim 73, wherein the nucleophile is an acetate salt.

75. The process of claim 74, wherein the acetate salt is selected from the group consisting of tetrabutyl ammonium acetate and potassium acetate.

76. The process of claim 59, wherein the resolving enzyme is selected from the group consisting of lipases, proteases, peptidases, amidases, acylases, and esterases.

77. The process of claim 76, wherein the resolving enzyme is a lipase.

78. The process of claim 59, wherein the compound of formula (III) reacts with an acetate in the presence of a solvent.

79. The process of claim 78, wherein the solvent is selected from the group consisting of t-butyl methyl ether and acetonitrile.

80. The process of claim 59, wherein the compound of formula (VI) is converted to the compound of formula (II) by deacetylation.

81. The process of claim 80, wherein the deacetylation is conducted in the presence of a base.

82. The process of claim 81, wherein the base is selected from the group consisting of alkali metal hydroxides, tetraalkylammonium hydroxides, and combinations thereof.

83. The process of claim 82, wherein the base is a mixture comprising potassium hydroxide and tetrabutylammonium hydroxide.

84. The process of claim 59, wherein the compound of formula (IV) is converted to the compound of formula (II) by alcoholysis.

85. The process of claim 84, wherein the alcoholysis is conducted in the presence of an alcohol selected from the group consisting of (C₁-C₆) alkanols.

86. The process of clam 84, wherein the alcoholysis is conducted in the presence of a base.

87. The process of claim 86, wherein the base is selected from the group consisting of alkali metal carbonates.

88. The process of claim 87, wherein the alkali metal carbonate is NaHCO₃ or KHCO₃.

89. The process of claim 59, wherein the acetate is selected from the group consisting of alkyl and alkenyl acetates.

90. The process of claim 89, wherein the alkenyl acetate is vinyl acetate.

91. The process of claim 59, wherein said process is a one-pot process.
